(19) United States
(12) Patent Application Publication
(10) Pub. No.: US 2026/0191604 A1
(43) Pub. Date: Jul. 9, 2026

(54) SURGICAL NAVIGATION SYSTEM AND NAVIGATION METHOD

(71) Applicant: B. Braun New Ventures GmbH, Freiburg im Breisgau (DE)

(72) Inventors: Amir Sarvestani, Freiburg (DE); Peter Heinze, Karlsruhe (DE)

(21) Appl. No.: 19/132,447

(22) PCT Filed: Nov. 16, 2023
(86) PCT No.: PCT/EP2023/082092

§ 371 Date: May 23, 2025

(30) Foreign Application Priority Data
Nov. 24, 2022 (DE) 10 2022 131 177.5

(51) Int. Cl. A61B 34/00; A61B 34/10; A61B 34/20; A61B 34/30; G16H 30/40

(52) U.S. Cl. CPC A61B 34/25; A61B 34/30; G16H 30/40; A61B 2034/107; A61B 2034/2055; A61B 2034/2065

(57) ABSTRACT

A surgical navigation system includes a first acquisition modality for acquiring a predefined region of tissue as a first recording with recording position data, and a second acquisition modality adapted to acquire the predefined region of the tissue as a second recording with recording position data. A memory unit stores a surgical map, with both a first numerical pathological value and a second numerical functional value of the tissue being stored for each position. A control unit analyzes the first and second recordings and assigns pathological and/or a functional values to portions of the tissue in the first and second recordings. The control unit visually outputs a view of the surgical map for navigation assistance purposes via a display device. The system can be used in a navigation method that is executable with the aid of a computer-readable storage medium and computer program.

### CROSS-REFERENCE TO RELATED APPLICATIONS

This application is the United States national stage entry of International Application No. PCT/EP2023/082092, filed on Nov. 16, 2023, and claims priority to German Application No. 2022 131 177.5, filed on Nov. 24, 2022. The contents of International Application No. PCT/EP2023/082092 and German Application No. 10 2022 131 177.5 are incorporated by reference herein in their entireties.

### FIELD

The present disclosure relates to a surgical navigation system for intraoperative guidance, such as of a surgical instrument, during a surgical intervention of tumor removal in a patient using a surgical map. For this purpose, the navigation system has a first acquisition modality, in particular a CT image device and/or an MRI image device, which is adapted to acquire, on the basis of a first acquisition modality, at least one predefined region of a tissue of the patient preoperatively and/or intraoperatively as a first image with image-position data and to provide it in a computer-readable manner. This means that a position of the image relative to a patient is also available as data for the first image, so that the image can also be assigned spatially relative to the patient. For example, in the case of an MRI image, the position of the tissue is also provided as image-position data. In addition, the present disclosure relates to a navigation method for intraoperative guidance, a computer-readable storage medium and a computer program.

### BACKGROUND

In brain surgery, neurosurgeons are very often faced with the decision to determine the boundaries of pathological tissue, as in particular tumor tissue, in order to remove as much tumor as possible while preserving as much healthy tissue (functional tissue) as possible and minimizing functional damage.

For this purpose, various modalities are used intraoperatively to either identify pathological tissue or to recognize critical structures (functional tissue) that need to be protected.

With the help of magnetic resonance imaging images or computer tomography images (MRI/CT data), for example, both the pathological and the critical structures to be preserved during the intervention can be identified. The same applies to intraoperative microscopy images/pictures. Recognition can be performed automatically, for example by a trained AI system, or manually by a doctor.

Fluorescence (imaging), such as 5-ALA, is used to identify tumor tissue, i.e. pathological tissue.

Intraoperative electrophysiologic signals, on the other hand, are used to identify important functional tissue to be preserved, such as important nerves.

Intraoperative histologic data taken from specimens are used to identify (selectively or area by area) both pathologic and healthy tissue, depending on the results of the analysis.

Unfortunately, the above-mentioned imaging procedures such as MRI/CT, microscopy or fluorescence have the disadvantage that they are not always precise in differentiating between pathological and healthy tissue. In addition, these methods have the disadvantage that they are generally not spatially assigned or correlated, i.e. they are not made available in a summarizing map. Electrophysiological data and histological data are more precise and differentiated, but are only available for individual regions of the anatomy. These limitations mean that the surgeon does not receive a homogeneous image of the surgical field in order to be able to reliably differentiate between pathological and healthy tissue.

Although surgeons use various modalities, there is still uncertainty as to where the boundaries of pathological tissue are actually to be defined.

### SUMMARY

Therefore, the objects and objectives of the present disclosure are to avoid or at least reduce the disadvantages of the prior art and in particular to provide a navigation system, a navigation method, a computer-readable storage medium as well as a computer program which provides visual assistance in the removal of pathological tissue, in particular tumor removal, in order to show the surgeon safely and intuitively a region of resection and a region of special care, for example a region of functional, vital tissue such as nerves, in order to enable safe navigation and resection. Another partial object may be to display a boundary line of a resection directly in a view in order to visually show possible incision lines and thus to support the surgeon.

Thus, a basic idea of the present disclosure is to provide a navigation system that utilizes various modalities such as microscopy, MRI images, CT images, electrophysiology and/or histology (or histological examinations) to create a central, homogeneous surgical map comprising areas/regions/zones with pathological tissue to be removed and tissue to be preserved (functional tissue; anatomy to be preserved) and defined with these and, in particular, together with calculated boundary lines, providing the user with a visual view of this surgical map to assist him/her intraoperatively.

While each individual modality is already known on its own, but as described in the introduction, this results in the corresponding disadvantages of an imprecise delimitation of regions, a solution is now provided here to combine all available modalities and to unite them in a single central surgical map that can define the boundaries of pathological tissue to be removed and healthy (functional) tissue to be preserved and can visually represent them to a surgeon. By combining data centrally and converting the information into a uniform, homogeneous ‘metric’ of the surgical map using the two position-related numerical values in a pathological-functional manner, such a central map can be created that intuitively presents a view with superimposed pathological tissue areas and functional tissue areas to the surgeon, for example by superimposing this surgical map with other images such as MRI images.

The term ‘image’ is to be understood quite broadly and means a punctual or small area image, for example via a biopsy at a position (the image position) and a corresponding histological assessment as to whether a pathological tissue or a functional tissue is present, or also a relatively large image, for example an MRI image of the patient, wherein the respective (three-dimensional) image-position data is of course also available for the MRI image. In other words, the position of the image (or the recorded tissue) relative to the patient is also recorded and provided for each image, so that the various acquisition modalities in the (single) central surgical map can also be added positionally correctly by corresponding determination of functional and pathological values.

The analysis and determination of whether a functional tissue or a pathological tissue is present can be carried out in different ways. In particular, such an analysis and determination may be performed automatically, in a computer-assisted manner. For example, a trained AI system that is trained with tumors may be adapted to recognize a tumor in an image, such as a CT image, and to identify this three-dimensional region as pathological tissue and to assign a pathological value to this pathological tissue. For example, the pathological value may be defined in a region from 0 to 10 and the functional value in a region from −10 to 0. In this way, an intensity of a pathological tissue may also be defined.

What is important here is that the navigation system of the present disclosure converts the many different acquisition modalities into the uniform ‘system’ with the pathological or functional values at the respective positions (relative to the patient) in order to generate a homogeneous database of pathological and functional tissue. This uniform database can then be used for further calculations and visualizations, such as a (standardized) calculation of a resection line to support the surgeon during resection.

In other words, the present disclosure relates to a navigation system that integrates various surgical modalities into a central computerized system. These modalities include in particular an MRI/CT (navigation), a microscopy, an endoscopy, a fluorescence, an electrophysiological detection and/or an intraoperative histology (e.g. via a Raman spectroscopy). The navigation system may also have a tracking system (e.g. an optical tracking system or a (robotic) kinematic tracking system) in order to determine the exact position of the corresponding modality data/modality images in or in relation to (the anatomy of) the patient. In addition to the image itself, the spatial image position may also be provided. Once the images or data of the modalities used have been acquired and integrated into the navigation system, a surgical map can be created. This map contains an intensity map that defines an intensity (first numerical pathological value, second numerical functional value) either in a 2D image/2D view, in particular for certain pixels (2D), or in a 3D space, in particular for certain voxels (3D). There are therefore two opposing/contrasting values or signal types. One is the pathological intensity and the other is the functional intensity. The more pathological a certain pixel or voxel is (in particular a tumor), the higher the pathological intensity. The more important a tissue is for preservation (e.g. an important nerve), the higher the functional intensity.

In particular, a (computer-aided) system with a display device and interfaces to different modalities may be provided, wherein position information for each modality is provided by a tracking system of the navigation system in order to be able to provide the (spatial) image positions in addition to the images, wherein a surgical map is created in two-dimensional form (2D) or three-dimensional form (3D) and in particular a color map is used to visualize how pathological or how functional the tissue is in order to determine the boundaries for the resection.

In yet other words, a surgical navigation system is provided for intraoperative guidance of a surgical instrument in a surgical intervention of tumor removal in a patient using a surgical map, comprising: a first acquisition modality, in particular a CT image device or an MRI image device, which is adapted to acquire, on the basis of a first (acquisition) modality, at least one predefined region of a tissue of the patient preoperatively and/or intraoperatively as a first image with image-position data and to provide it in a computer-readable manner; a second acquisition modality different from the first acquisition modality, in particular fluorescence imaging or an electrophysiological image, which is adapted to acquire, on the basis of a second (acquisition) modality, the predefined region of the tissue of the patient preoperatively and/or intraoperatively as a second image with image-position data and to provide it in a computer-readable manner; a storage unit in which the two-dimensional or three-dimensional surgical map of the patient is stored, wherein for each (2D or 3D) position (pixel or voxel) of the surgical map, at least in the predefined region of the tissue, a first numerical pathological value as well as a second numerical functional value of the tissue is stored; a control unit adapted to: —process the provided first image with image-position data and the provided second image with image-position data, —for the first acquisition modality, to analyze and determine the first image in such a way that a pathological value is assigned to at least a partial region of the tissue of the first image, and/or a functional value is assigned to at least a partial region of the tissue of the first image, and to add these pathological and functional values, respectively, to the corresponding position of the surgical map; —for the second acquisition modality, to analyze and determine the second image in such a way that a pathological value is assigned to at least a partial region of the tissue of the second image, and/or a functional value is assigned to at least a partial region of the tissue of the second image, and to add these pathological or respectively functional values in the associated position of the surgical map; and —to visually output a view of the surgical map for navigation assistance via a display device, in particular an operating room monitor.

Advantageous embodiments are explained in particular below.

In particular, the navigation system may be adapted to register the first images (of the first acquisition modality) in the form of CT images and/or MRI images and/or DTI data by tracking by a tracking system of the navigation system with the patient; and
to spatially track a position of a surgical microscope (as visualization system and further acquisition modality with corresponding second (microscope) image) and/or an endoscope (as visualization system and further acquisition modality with corresponding second (endoscope) image) by the navigation system; and/or
to detect and localize electrophysiological images or signals with a tracked probe through the navigation system; and/or
to detect and spatially localize histological images or data with a tracked (biopsy) probe using the navigation system. This makes it possible to provide different images with the corresponding image-position data, which can be transferred to the surgical map.

In particular, the navigation system may comprise a robot or a robotic system (combined with a robotic system), wherein the visualization system (in particular surgical microscope and/or endoscope) and/or the probes are moved and located with a robotic arm of the robotic system. In particular, the robot kinematics may be used as a tracking system to determine the position of the visualization system and/or of the probe.

According to one embodiment, the control unit may be adapted to assign a first color to the first numerical pathological value and to assign a second, different, in particular complementary, color to the second numerical functional value of the tissue, wherein an intensity of the color is proportional to the numerical value, so that when the view is output via the display device, a surgical map with a color background is output. Colors may therefore be used to differentiate between pathological and functional intensity. In particular, a pathological intensity may be displayed in red and a functional intensity in blue. In particular, the colors of the pathological values are complementary colors to the functional values. As a result, a color map is created as a surgical map, where a higher intensity of red means that more resection is recommended, and blue means that less resection is recommended. Since no data (such as electrophysiological signals or histology data) is available for each pixel or voxel for non-image-based modalities, the color mapping allows extrapolation of the labeling of pathological and functional regions based on a 3D point/position signal. With this visualization, the surgeon can make a better choice on where to resect tissue during surgery. Based on the data of the central surgical map, a view may also be individually adjusted. In particular, such a view can be created on the basis of punctual (position-related) values, in which an extrapolation takes place between the individual values in order to display a continuously differentiable surface.

According to a further embodiment, the control unit may be adapted to determine a gradient between the pathological value and the functional value and to display a boundary line in the view based on the gradient in order to show the user a boundary for a resection. In particular, the navigation system may therefore be adapted to generate concrete boundary lines (in the case of 2D) or boundary (surface) areas (in the case of 3D) using image gradients. For example, between a red, pathological region of the tissue and a blue, functional region of the tissue, the position with the highest gradient is used to define the boundaries. As a result, not only color maps indicating pathological and functional tissue, but also boundaries for resection may be defined and displayed. The navigation system may therefore also use image gradients to calculate and visualize clear boundaries.

Preferably, the navigation system may comprise an input unit, in particular the display device may be configured as a touch display to detect an input by the user, wherein the control unit is adapted to adjust a gradient setting based on the input in order to change a boundary of the resection in the view of the surgical map. In particular, a control (such as a slider) for a gradient setting may be shown on the touch display and the user may then change the setting by touching this control. Depending on the aggressiveness of the pathology (e.g. glioblastoma, where aggressive resection is recommended), the gradient may therefore be adjusted to meet the surgeon's preferences and the patient's needs. The adjustable gradient allows a more or less aggressive resection depending on the type of pathology. In particular, the navigation system may also use adjustable gradients in order to define the aggressiveness of the resection region. In particular, the user may use the input to either move or adjust the boundary in a ‘plus direction’ (i.e. more pathology) from a maximum of the gradient or gradients in order to protect more functional tissue, or move or adjust the boundary in a ‘minus direction’ (i.e. more function) from the maximum of the gradient in order to remove pathological tissue more aggressively. In particular, it is therefore possible to adjust the aggressiveness of the tissue removal by ‘shifting’ the boundary from a maximum of the gradient in a ‘plus direction’ (i.e. a further/larger boundary around or in relation to the functional tissue) or in a ‘minus direction’ (i.e. a smaller boundary around or in relation to the functional tissue). The gradient shows its maximum where the greatest difference between pathological and functional tissue is. If, for example, an initial boundary is drawn at a maximum of the gradients, this boundary may be adjusted in the direction of the functional tissue for more aggressive removal or in the direction of the pathological tissue for less aggressive removal.

Preferably, the navigation system may comprise an input unit, in particular the display device may be configured as a touch display, to detect an input by the user, wherein the control unit is adapted to manually define regions of a tissue based on the input, to which a first numerical pathological value and/or a second numerical functional value of the tissue is assigned via an input to also manually identify regions. In addition to the at least two different acquisition modalities, surgeons may also manually define regions with important function or pathology that may be added to the surgical map, or additional regions may be added using image segmentation.

In particular the following may be used as at least the first or second acquisition modality:
an MRI image and/or a CT image and/or a DTI image;
a surgical microscope;
an endoscope;
an electrophysiology;
a histology; and/or
a fluorescence imaging device.

The modalities may therefore include in particular: CT/MRI/DTI with navigation, microscope, endoscope, electrophysiology, histology, fluorescence imaging. These different acquisition modalities each have the advantages of a precise determination of pathological or functional tissue and can be used in a targeted manner to combine and unite all the advantages.

In particular, the first and/or second acquisition modality may be connected as an end effector to a robot, in particular a surgical microscope and/or an endoscope and/or a fluorescence imaging device may be mounted as an end effector on a robot arm. In other words, the microscope, the endoscope and/or the fluorescence imaging device (imager) may be mounted on a robot arm and may be activated and moved or positioned by the robot arm.

Preferably, a probe that measures data, such as a biopsy probe, may be attached to the robotic arm.

According to one embodiment, the navigation system may have a navigation camera as an optical camera and may be adapted to track fiducial trackers, or the navigation system may have a navigation camera as an optical camera and may use a machine-learning image-processing system in order to track objects spatially. In particular, the navigation system may use an optical (navigation) camera with fiducial trackers or a (machine) image processing system.

With regard to a navigation method for intraoperative guidance of a surgical instrument in a surgical intervention of tumor removal in a patient using a surgical map, the objects are solved by the steps of: —acquiring at least one predefined region of a tissue of the patient preoperatively and/or intraoperatively as a first image with image-position data via a first acquisition modality, in particular a CT image device or an MRI image device; —acquiring the predefined region of the tissue of the patient preoperatively and/or intraoperatively as a second image with image-position data with a second acquisition modality different from the first acquisition modality, in particular fluorescence imaging or an electrophysiological image; —analyzing and determining for the first acquisition modality of the first image in such a way that a pathological value is assigned to at least a partial region of the tissue of the first image, and/or a functional value is assigned to at least a partial region of the tissue of the first image, and adding these pathological or functional values in the associated position of a surgical map in which a first numerical pathological value and a second numerical functional value of the tissue are stored for each (2D or 3D) position (pixel or voxel) at least in the predefined region of the tissue; —analyzing and determining for the second acquisition modality of the second image that a pathological value is assigned to at least a partial region of the tissue of the second image, and/or a functional value is assigned to at least a partial region of the tissue of the second image, and adding this pathological or functional value in the associated position of the surgical map; and —outputting via a display device, in particular a surgical monitor, a view of the surgical map for navigation assistance.

With respect to a computer-readable storage medium and a computer program, the objects are solved by comprising instructions which, when executed by a computer, cause the computer to execute the method steps of the navigation method according to the present disclosure.

The application in brain surgery described here may of course also be used for other indications where different modalities are used intraoperatively to differentiate the boundaries between pathological and functional tissue.

### BRIEF DESCRIPTION OF THE DRAWINGS

The present disclosure is explained in more detail below with reference to the accompanying Figures, with reference to preferred embodiments. The following is shown:

FIG. 1 shows a schematic view of a navigation system of a first embodiment of the present disclosure with different acquisition modalities;

FIG. 2 shows a schematic view of a combination of pathological values and functional values to define the color maps and to calculate the resection boundaries;

FIG. 3 shows an exemplary surgical map with adjustable boundaries or boundary lines;

FIG. 4 shows an exemplary surgical map as a color map with red and blue intensity maps, wherein red is used for pathological tissue and blue for important functional tissue;

FIG. 5 shows a two-dimensional plan view of the surgical map of FIG. 5; and

FIG. 6 shows a schematic flow diagram of a navigation method according to a preferred embodiment.

The Figures are schematic in nature and are intended only to aid understanding of the present disclosure. Identical elements are provided with the same reference signs. The features of the various configuration examples can be interchanged.

### DETAILED DESCRIPTION

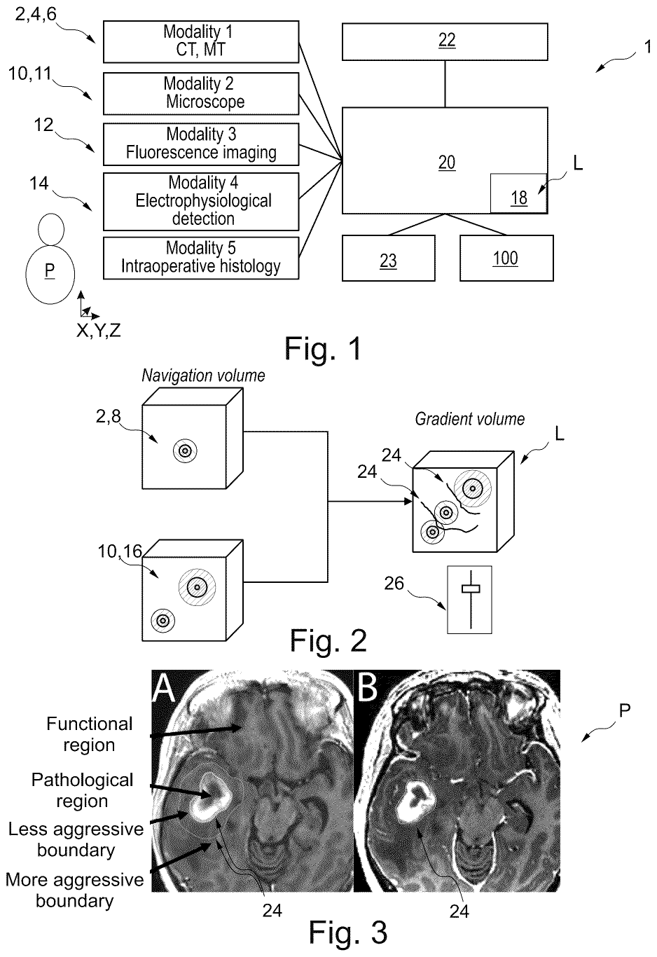

FIG. 1 shows a schematic view of a surgical navigation system 1 (hereinafter referred to as system) for intraoperative guidance of a surgical cutting instrument during a surgical intervention to remove a tumor from a patient P using a surgical map L.

The system 1 has a first acquisition modality 2 in the form of a CT image device 4 and also an MRI image device 6 and is adapted to acquire at least one predefined region of a tissue of the patient P preoperatively as a first image 8 with associated image-position data, i.e. information on the spatial position of the image in relation to the patient P, and to provide it in a computer-readable manner on the basis of this first acquisition modality.

In addition, the system 1 also has a further second acquisition modality 10, different from the first acquisition modality 2, in the form of a surgical microscope 11 and is adapted to acquire the predefined region of the tissue of the patient P preoperatively and/or intraoperatively as a second image 16 with image-position data and to provide it in a computer-readable manner on the basis of the second (acquisition) modality.

The system 1 also has a further, third acquisition modality in the form of fluorescence imaging 12. With this acquisition modality, the image is also performed in the same way as with the first and second acquisition modality 2, 10.

In addition, the system also has a fourth acquisition modality in the form of an electrophysiological image system 14 and a fifth acquisition modality in the form of intraoperative histology, i.e. intraoperative sampling using a probe.

All of these five different acquisition modalities are brought together and processed centrally. For this purpose, the system has a storage unit 18, in which the two-dimensional or three-dimensional surgical map L of the patient P is stored, wherein for each (2D or 3D) position (pixel or voxel) of the surgical map L, at least in the predefined region of the tissue, a first numerical pathological value and a second numerical functional value of the tissue are stored or can be stored. In this embodiment, a three-dimensional surgical map L is stored, wherein a pathological value (P-value) and a functional value (F-value) are stored for each position (X, Y, Z) (here set to 0 before the images). This allows the system 1 to read out an intensity of a pathological tissue and a functional tissue after an image and corresponding addition of the respective pathological or functional values for a specific position (x1, y1, z1).

Furthermore, the system 1 comprises a central control unit 20 adapted: to process the provided first image 8 with image-position data and the provided second image 16 with image-position data. Likewise, the control unit 20 is also adapted to process the third image of the third acquisition modality, the fourth image of the fourth acquisition modality and the fifth image of the acquisition modality with corresponding image-position data. A tracking system 23 may also be used to determine the position of the acquisition modality and thus of the image itself (for example via a transformation).

The control unit 20 is further adapted to analyze and determine the first image 8 for the first acquisition modality 2 in such a way that a pathological value is assigned to at least a partial region of the tissue of the first image 8, and/or a functional value is assigned to at least a partial region of the tissue of the first image, and to add these pathological or respectively functional values in the associated position of the surgical map L. In this embodiment, the detected pathological value (here positive) is added to the pathological value of the surgical map depending on the position and the detected, here negative, functional value of the surgical map is also added. In particular, the MRI image may be analyzed by an artificial intelligence system (AI system) trained by tumors, which determines the regions of pathological tissue in the MRI image and evaluates them according to the pathological values. In particular, the control unit 20 performs the analysis and determination and is adapted accordingly.

Likewise, for the second acquisition modality 10, the second image is analyzed and determined in such a way that a pathological value is assigned to at least a partial region of the tissue of the second image and/or a functional value is assigned to at least a partial region of the tissue of the second image, and these pathological or functional values are added to the associated position of the surgical map L.

Similarly, for the third, fourth and fifth acquisition modalities, a pathological and/or functional value is assigned for various respective spatial positions (when analyzed and determined) and is added to the central (single) surgical map L in each case.

This results in a central surgical map L, in which the various modalities are combined and integrated to provide a uniform, homogeneous, central map L (see FIG. 2, for example), which may be integrated into different output modes. This surgical map L may be superimposed on a three-dimensional MRI image, for example, in order to show the regions and boundaries and display them visually to a surgeon.

The control unit 20 of the present embodiment is adapted to visually output a view of the surgical map L for navigation assistance via a display device 22 in the form of a surgical monitor, for example a virtual perspective view at a certain position with a predetermined viewing direction of the tissue with marked regions of pathological tissue and functional tissue and a displayed boundary line 24 (see also FIG. 2).

In contrast to the state of the art, an individual acquisition modality is not considered separately and in isolation, but the many different acquisition modalities are all used and centrally merged, standardized and stored in the central surgical map L.

In particular, extrapolations may also be performed to estimate the surrounding region from a punctual value, such as a pathological value determined with the aid of a biopsy.

FIG. 2 shows in a schematic view for the understanding of the present disclosure the combination of pathological values and functional values into a standardized map L comprising both values to support navigation.

At the top left of FIG. 2, a so-called antagonist of a cold zone is shown as an example, which represents a risk structure and therefore has a high functional value. This risk structure may be determined in particular via a first acquisition modality 2 using in particular DTI/fibers, segmentations of for example MRI images or CT images as well as neuromonitoring. This provides the system 1 with a functional map of a risk structure (i.e. an anatomy to be preserved).

On the other hand, a so-called protagonist of a hot zone, which represents the target structure of the resection, is shown at the bottom left of FIG. 2 as an example. This data may be obtained by a further, second acquisition modality 10, here for example an MRI image with delineation of a tumor or a biopsy/histology result or fluorescence imaging in order to make tumor structures visible and to detect them. This provides the system 1 with a pathological map of a structure to be removed.

Both maps, i.e. the functional map and the pathological map, are merged and combined in the central surgical map L. This means that both functional structures and pathological structures are integrated and centrally available. If further acquisition modalities are added, these new acquisition modalities may simply supplement the central surgical map with their information on functional or pathological tissue, positionally correct in relation to the patient (after all, the images have the image-position data). A dynamically growing surgical map L may therefore be created that brings together and combines all information centrally and homogeneously.

Finally, to support the surgeon in the intervention, a so-called gradient volume, as shown in the right part of FIG. 2, may be output, in which a gradient between the functional region and the pathological region is output in a view, for example via the surgical monitor. Boundary lines 24 are already indicated here, which represent a standardized calculated resection boundary.

Using a slider as input unit 26, for example shown in a touch display and also operable, a gradient setting may be changed in order to adjust the aggressiveness of the resection by changing the resection boundaries or the boundary lines 24.

A threshold value may therefore be set on gradient-defined contours (level sets). This may serve as a guide for the surgeon in particular. For example, a steep or high gradient may be difficult to distinguish because the pathological and functional regions are close together.

FIG. 3 shows an example of such a gradient setting for illustration purposes. While a gradient between a functional region and a pathological region is shown schematically in region A of FIG. 3, two different boundaries are visible here. A first boundary or boundary line 24, which indicates a less aggressive resection of the tumor tissue (pathological tissue) and a second boundary of an aggressive resection in order to detect and remove a region around the pathological tissue. The gradient may therefore be set between these two boundary lines 24 of different aggressiveness. In region B of FIG. 3, a boundary of a very defensive resection is shown as an example.

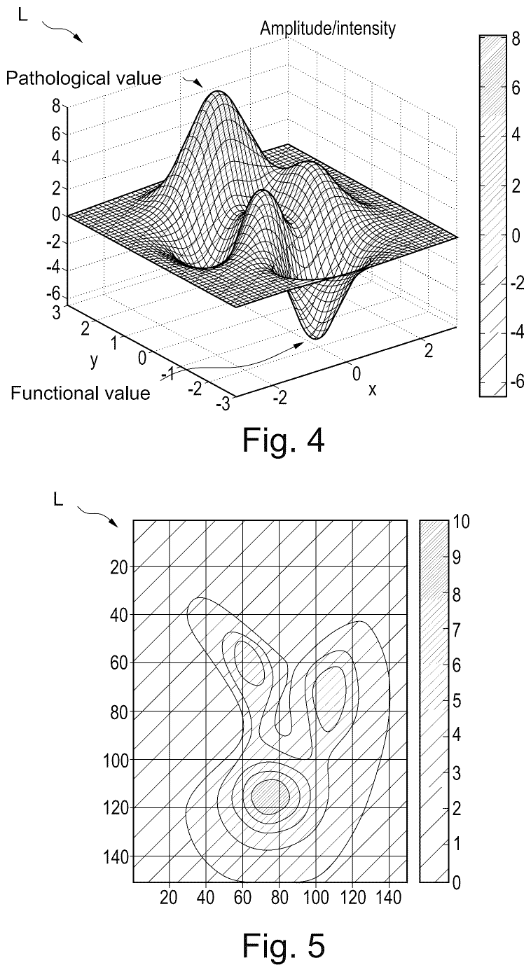

FIG. 4 shows an exemplary representation of a surgical map L as a color map with red and blue intensity maps, wherein red is used for pathological tissue and blue for important functional tissue (shown here with different shading for the colors). The amplitudes represent the regions accordingly. Positive amplitudes represent the pathologic region with the pathologic values, while negative amplitudes represent the functional region to be spared. A continuous surface is shown as an example between these regions. Gradients may be used to set a boundary.

FIG. 5 is an exemplary top view of FIG. 4 and shows in two-dimensional form the regions to be removed (red).

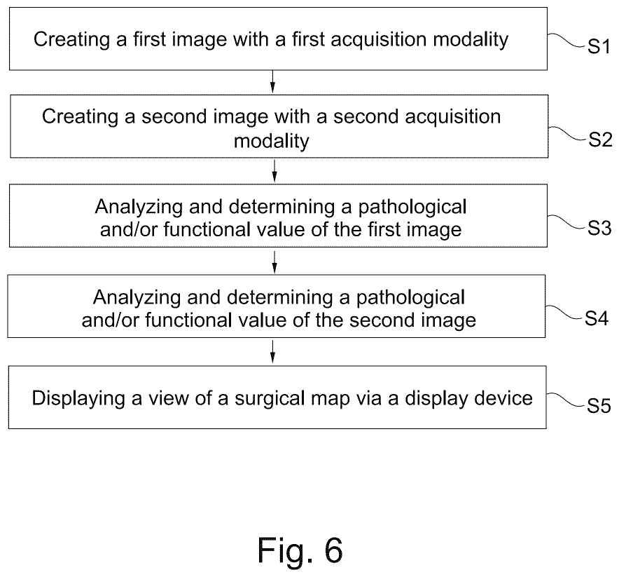

FIG. 6 shows a navigation method according to a preferred embodiment. The navigation method is used for intraoperative guidance of a surgical instrument during a surgical intervention of tumor removal in a patient using a surgical map, in particular for a navigation system 1 of the present disclosure.

In step S1, at least one predefined region of a tissue of the patient P is detected preoperatively and/or intraoperatively as a first image with image-position data via a first acquisition modality, in particular a CT image device or an MRI image device;

In step S2, the predefined regions of the patient's tissue are then detected preoperatively and/or intraoperatively as a second image with image-position data with a second acquisition modality different from the first acquisition modality, in particular fluorescence imaging or an electrophysiological image;

In step S3, the analyzing and determining is carried out for the first acquisition modality of the first image in such a way that a pathological value is assigned to at least a partial region of the tissue of the first image and/or a functional value is assigned to at least a partial region of the tissue of the first image, and these pathological or respectively functional values are added to the associated position of a surgical map, in which a first numerical pathological value and a second numerical functional value of the tissue are stored for each position at least in the predefined region of the tissue.

In step S4, the analyzing and determining is carried out for the second acquisition modality in such a way that a pathological value is assigned to at least a partial region of the tissue of the second image and/or a functional value is assigned to at least a partial region of the tissue of the second image, and this pathological or functional value is added to the associated position of the surgical map.

Finally, in a step S5, a view of the surgical map for navigation assistance is output via a display device, in particular a surgical monitor.

### LIST OF REFERENCE SIGNS

1 surgical navigation system
2 first acquisition modality
4 CT image device
6 MRI image device
8 first image
10 second acquisition modality
11 surgical microscope
12 fluorescence imaging
14 electrophysiological image
16 second image
18 storage unit
20 control unit
22 display device
23 tracking system
24 boundary line
26 input unit
100 robot
P patient
L surgical map
S1 Step acquiring with first acquisition modality
S2 Step acquiring with second acquisition modality
S3 Step analyzing and determining pathological and/or functional value of first image
S4 Step analyzing and determining pathological and/or functional value of second image
S5 Step outputting view of surgical map via display device

1.-12. (canceled)

13. A surgical navigation system for intraoperative guidance of a surgical instrument in a surgical intervention of tumor removal in a patient using a surgical map, the surgical navigation system comprising:
a first acquisition modality adapted to acquire at least one predefined region of a tissue of the patient preoperatively and/or intraoperatively as a first image with image-position data to detect and to provide the first image that is computer-readable;
a second acquisition modality different from the first acquisition modality, the second acquisition modality adapted to acquire the at least one predefined region of the tissue of the patient preoperatively and/or intraoperatively as a second image with image-position data and to provide the second image that is computer-readable;
a storage unit in which a surgical map, which is a central two-dimensional or three-dimensional surgical map of the patient, is stored, wherein for each position of the surgical map, at least in the at least one predefined region of the tissue, a first numerical pathological value as well as a second numerical functional value of the tissue is stored or storable; and
a control unit that is adapted to:
process the first image with image-position data and the second image with image-position data,
analyze and determine the first image, for the first acquisition modality, in such a way that a first pathological value and/or functional value is assigned to at least a partial region of the tissue of the first image, and to add the first pathological value and/or functional value in an associated position of the surgical map,
analyze and determine the second image, for the second acquisition modality, in such a way that a second pathological value and/or functional value is assigned to at least a partial region of the tissue of the second image, and to add the second pathological value and/or functional value in an associated position of the surgical map, and
visually output a view of the surgical map for navigation assistance via a display device in order to reliably distinguish between a pathological tissue and a functional tissue.

14. The surgical navigation system according to claim 13, wherein the first acquisition modality comprises a CT image device or an MRI image device.

15. The surgical navigation system according to claim 13, wherein the second acquisition modality comprises fluorescence imaging or an electrophysiological image.

16. The surgical navigation system according to claim 13, wherein the display device comprises an operating room monitor.

17. The surgical navigation system according to claim 13, wherein the control unit is adapted to assign a first color to the first numerical pathological value and to assign a second color different from the first color to the second numerical functional value of the tissue, wherein the first color has a first intensity proportional to the first pathological value and/or functional value, and the second color has a second intensity proportional to the second pathological value and/or functional value so that when the view is output via the display device, a surgical map with a color background is output.

18. The surgical navigation system according to claim 13, wherein the control unit is adapted to determine a gradient between the pathological values and the functional value of the surgical map and to display a boundary line in the view based on the gradient in order to show a user a boundary for a resection.

19. The surgical navigation system according to claim 18, further comprising an input unit to detect an input by the user, wherein the control unit is adapted to adjust a gradient setting based on the input in order to change a boundary line of the resection in the view of the surgical map.

20. The surgical navigation system according to claim 19, wherein the input unit comprises a touch display on the display device.

21. The surgical navigation system according to claim 13, further comprising an input unit to detect an input by a user, wherein the control unit is adapted to manually define regions of a tissue based on the input, to which a first numerical pathological value and/or a second numerical functional value of the tissue is assigned via the input in order to also manually identify regions.

22. The surgical navigation system according to claim 21, wherein the input unit comprises a touch display on the display device.

23. The surgical navigation system according to claim 13, wherein the following is used as at least the first acquisition modality or the second acquisition modality:
an MRI image device and/or a CT image device and/or a DTI image device;
a surgical microscope;
an endoscope;
electrophysiology;
a histology; and/or
a fluorescence imaging device.

24. The surgical navigation system according to claim 13, wherein the first acquisition modality and/or second acquisition modality is connected to a robot as an end effector.

25. The surgical navigation system according to claim 24, wherein:
the end effector comprises at least one of a surgical microscope, an endoscope, and a fluorescence imaging device, and
the end effector is mounted on a robot arm.

26. The surgical navigation system according to claim 13, further comprising a robot having a robotic arm, the robotic arm being attached to probes that measure data.

27. The surgical navigation system according to claim 13, further comprising a navigation camera as an optical camera, wherein:
the navigation camera is adapted to track fiducial trackers, or
the navigation camera uses a machine image-processing system to track objects spatially.

28. A navigation method for intraoperative guidance of a surgical instrument in a surgical intervention of tumor removal in a patient using a surgical map, the navigation method comprising the steps of:
acquiring at least one predefined region of a tissue of the patient preoperatively and/or intraoperatively as a first image with image-position data via a first acquisition modality;
acquiring the at least one predefined region of the tissue of the patient preoperatively and/or intraoperatively as a second image with image-position data with a second acquisition modality different from the first acquisition modality;
analyzing and determining for the first acquisition modality of the first image in such a way that a first pathological value and/or a functional value is assigned to at least a partial region of the tissue of the first image, and adding the first pathological value and/or a functional value in an associated position of a surgical map, in which a first numerical pathological value as well as a second numerical functional value of the tissue is stored or can be stored for each position at least in the at least one predefined region of the tissue;
analyzing and determining for the second acquisition modality of the second image that a second pathological value and/or a functional value is assigned to at least a partial region of the tissue of the second image, and adding the second pathological value and/or a functional value in an associated position of the surgical map; and
outputting via a display device a view of the surgical map for navigation assistance to distinguish between a pathological tissue and a functional tissue.

29. The navigation method according to claim 28, wherein the first acquisition modality comprises a CT image device or an MRI image device.

30. The navigation method according to claim 28, wherein the second acquisition modality comprises fluorescence imaging or an electrophysiological image;

31. A computer-readable storage medium comprising instructions which, when executed by a computer, cause the computer to execute the navigation method according to claim 28.

32. A computer program comprising instructions which, when executed by a computer, cause the computer to execute the navigation method according to claim 28.
